(19) United States
(12) Patent Application Publication
(10) Pub. No.: US 2024/0021299 A1
(43) Pub. Date: Jan. 18, 2024

(54) MEDICAL SYSTEMS AND METHODS FOR MOVABLE MEDICAL DEVICES

(71) Applicant: SHANGHAI UNITED IMAGING HEALTHCARE CO., LTD., Shanghai (CN)

(72) Inventor: Jie YU, Shanghai (CN)

(73) Assignee: SHANGHAI UNITED IMAGING HEALTHCARE CO., LTD., Shanghai (CN)

(21) Appl. No.: 18/353,855

(22) Filed: Jul. 17, 2023

(30) Foreign Application Priority Data
Jul. 15, 2022 (CN) 202210832228.1

(51) Int. Cl. G16H 40/40; G16H 40/20; G16H 10/60; A61B 6/04

(52) U.S. Cl. CPC G16H 40/40; G16H 40/20; G16H 10/60; A61B 6/0407

(57) ABSTRACT

A medical system for movable medical devices may be provided. First information relating to an examination room where a movable medical device is located may be provided. Further, at least one of environment information of the examination room or subject information relating to one or more subjects in the examination room may be determined based on the first information. The one or more subjects may include a target subject to be scanned. Then, a scan protocol to be used in a medical scan of the target subject may be determined based on the at least one of the environment information or the subject information.

### CROSS-REFERENCE TO RELATED APPLICATIONS

This application claims priority of Chinese Patent Applications No. 202210832228.1, filed on Jul. 15, 2022, the contents of which are incorporated herein by reference.

### TECHNICAL FIELD

The present disclosure relates to medical technology, and in particular, to medical systems and methods for movable medical devices.

### BACKGROUND

When using an immobile medical device, a patient needs to be moved to a specific examination room for medical scan, which is inconvenient in some occasions (e.g., medical scan of emergency patients, intraoperative evaluation, etc.). A movable medical device can be quickly moved to a position of the patient to complete the medical scan, which is very convenient and increasingly used in medical scans. Therefore, it is desirable to provide medical systems and methods for movable medical devices medical scan.

### SUMMARY

According to an aspect of the present disclosure, a medical system for movable medical devices may be provided. The system may include at least one storage device including a set of instructions and at least one processor in communication with the at least one storage device. When executing the set of instructions, the at least one processor may be configured to direct the system to perform the following operations. The system may obtain first information relating to an examination room where a movable medical device is located. The system may also determine at least one of environment information of the examination room or subject information relating to one or more subjects in the examination room based on the first information. The one or more subjects may include a target subject to be scanned. Further, the system may determine a scan protocol to be used in a medical scan of the target subject based on the at least one of the environment information or the subject information.

In some embodiments, the first information may include image information of the examination room, and the environment information of the examination room may be determined based on the image information of the examination room using a determination model. The determination model may be a trained machine learning model.

In some embodiments, the first information may include image information of the examination room, and the environment information of the examination room may be determined based on the image information of the examination room by performing the following operations. For each of one or more reference examination rooms, the system may obtain reference image information of the reference examination room and reference environment information corresponding to the reference examination room. The system may also generate a comparison result by comparing the image information with the reference image information of each reference examination room. Further, the system may determine the environment information of the examination room based on the comparison result and the reference environment information corresponding to each reference examination room.

In some embodiments, the first information may include position information of the movable medical device in a building where the examination room is located, and the environment information of the examination room may be determined based on the position information of the examination room by performing the following operations. The system may determine a position of the examination room in the building based on the position information of the movable medical device. The system may further determine the environment information of the examination room based on the position of the examination room.

In some embodiments, the scan protocol to be used in the medical scan of the target subject may be determined based on the environment information by performing the following operations. The system may obtain one or more one or more preset scan protocols corresponding to the examination room according to the environment information of the examination room. Further, the system may determine the scan protocol from the one or more preset scan protocols.

In some embodiments, the scan protocol to be used in the medical scan of the target subject may be determined based on the environment information by performing the following operations. The system may obtain second information relating to the target subject. The system may also determine a scanning region of the target subject that needs to receive the medical scan based on the second information. Further, the system may determine the scan protocol based on the environment information and the scanning region.

In some embodiments, to determine the scan protocol based on the environment information and the scanning region, the system may perform the following operations. The system may obtain one or more candidate scan protocols based on the environment information. The one or more candidate scan protocols may include one or more preset scan protocols corresponding to the examination room and/or one or more historical scan protocols of one or more historical medical scans corresponding to the examination room. Further, the system may select the scan protocol based on the scanning region from the one or more candidate scan protocols.

In some embodiments, to determine the scan protocol based on the environment information and the scanning region, the system may perform the following operations. The system may determine whether there are one or more preset scan protocols corresponding to the examination room. In response to determining that there are one or more preset scan protocols corresponding to the examination room, the system may determine the scan protocol based on the one or more preset scan protocols. In response to determining that there is no preset scan protocol, the system may determine the scan protocol based on the scanning region.

In some embodiments, the second information may include image information of the target subject, and the scanning region may be determined by processing the image using a region determination model. The region determination model may be a trained machine learning model.

In some embodiments, the second information may include identity information of the target subject collected by an identification device disposed on the movable medical device, and the scanning region may be determined based on the identity information of the target subject by retrieving a medical database.

In some embodiments, the subject information relating to one or more subjects may include identity information of the one or more subject collected by an identification device disposed on the movable medical device, and the scan protocol may be determined based on the identity information of the one or more subjects by retrieving a medical database.

In some embodiments, the scan protocol to be used in the medical scan of the target subject may be determined based on the environment information by performing the following operations. The system may determine whether there are a plurality of preset scan protocols correspond to different regions in the examination room. In response to determining that there are a plurality of preset scan protocols correspond to different regions in the examination room, the system may further determine the scan protocol based on a region of the examination room where the movable medical device is located.

In some embodiments, the system may perform the following operations. The system may determine scan direction information of the medical scan based on the scan protocol. The scan direction information may include at least one of a moving direction of a table for supporting the target subject, a rotation direction of a radiation resource that performs the medical scan, or an angle between a rotation plane of the radiation resource and a surface of the table for bearing the target subject.

In some embodiments, to determine scan direction information of the medical scan based on the scan protocol, the system may perform the following operations. The system may determine a position of the movable medical device relative to the target subject. The system may determine the scan direction information of the medical scan based on the scan protocol and the position of the movable medical device relative to the target subject.

According to another aspect of the present disclosure, a method for movable medical devices may be provided. The method may include obtaining first information relating to an examination room where a movable medical device is located. The method may include determining. at least one of environment information of the examination room or subject information relating to one or more subjects in the examination room based on the first information. The one or more subjects may include a target subject to be scanned. Further, the method may include determining a scan protocol to be used in a medical scan of the target subject based on the at least one of the environment information or the subject information.

According to yet another aspect of the present disclosure, a non-transitory computer readable medium may be provided. The non-transitory computer readable medium may include at least one set of instructions for medical imaging. When executed by one or more processors of a computing device, the at least one set of instructions may cause the computing device to perform a method. The method may include obtaining first information relating to an examination room where a movable medical device is located. The method may include determining at least one of environment information of the examination room or subject information relating to one or more subjects in the examination room based on the first information. The one or more subjects may include a target subject to be scanned. Further, the method may include determining a scan protocol to be used in a medical scan of the target subject based on the at least one of the environment information or the subject information.

Additional features will be set forth in part in the description which follows, and in part will become apparent to those skilled in the art upon examination of the following and the accompanying drawings or may be learned by production or operation of the examples. The features of the present disclosure may be realized and attained by practice or use of various aspects of the methodologies, instrumentalities, and combinations set forth in the detailed examples discussed below.

### BRIEF DESCRIPTION OF THE DRAWINGS

The present disclosure is further described in terms of exemplary embodiments. These exemplary embodiments are described in detail with reference to the drawings. These embodiments are non-limiting exemplary embodiments, in which like reference numerals represent similar structures throughout the several views of the drawings, and wherein:

FIG. 1 is a schematic diagram illustrating an exemplary medical system according to some embodiments of the present disclosure;

FIG. 2 is a block diagram illustrating exemplary processing device according to some embodiments of the present disclosure;

FIG. 3 is a flowchart illustrating an exemplary process for determining a scan protocol to be used in a medical scan of a target subject according to some embodiments of the present disclosure;

FIG. 4 is a flowchart illustrating an exemplary process for determining a scan protocol to be used in a medical scan of a target subject according to some embodiments of the present disclosure;

FIG. 5 is a flowchart illustrating an exemplary process for determining scan direction information of a medical scan of a target subject according to some embodiments of the present disclosure; and

FIG. 6 provides a schematic diagram illustrating an exemplary process for scan protocol determination according to some embodiments of the present disclosure.

### DETAILED DESCRIPTION

In the following detailed description, numerous specific details are set forth by way of examples in order to provide a thorough understanding of the relevant disclosure. However, it should be apparent to those skilled in the art that the present disclosure may be practiced without such details. In other instances, well-known methods, procedures, systems, components, and/or circuitry have been described at a relatively high level, without detail, in order to avoid unnecessarily obscuring aspects of the present disclosure. Various modifications to the disclosed embodiments will be readily apparent to those skilled in the art, and the general principles defined herein may be applied to other embodiments and applications without departing from the spirit and scope of the present disclosure. Thus, the present disclosure is not limited to the embodiments shown, but to be accorded the widest scope consistent with the claims.

In the following detailed description, numerous specific details are set forth by way of examples in order to provide a thorough understanding of the relevant disclosure. However, it should be apparent to those skilled in the art that the present disclosure may be practiced without such details. In other instances, well-known methods, procedures, systems, components, and/or circuitry have been described at a relatively high level, without detail, in order to avoid unnecessarily obscuring aspects of the present disclosure. Various modifications to the disclosed embodiments will be readily apparent to those skilled in the art, and the general principles defined herein may be applied to other embodiments and applications without departing from the spirit and scope of the present disclosure. Thus, the present disclosure is not limited to the embodiments shown, but to be accorded the widest scope consistent with the claims.

The terminology used herein is for the purpose of describing particular example embodiments only and is not intended to be limiting. As used herein, the singular forms “a,” “an,” and “the” may be intended to include the plural forms as well, unless the context clearly indicates otherwise. It will be further understood that the terms “comprise,” “comprises,” and/or “comprising,” “include,” “includes,” and/or “including,” when used in this specification, specify the presence of stated features, integers, steps, operations, elements, and/or components, but do not preclude the presence or addition of one or more other features, integers, steps, operations, elements, components, and/or groups thereof.

It will be understood that the term “system,” “engine,” “unit,” “module,” and/or “block” used herein are one method to distinguish different components, elements, parts, sections or assembly of different levels in ascending order. However, the terms may be displaced by another expression if they achieve the same purpose.

Generally, the word “module,” “unit,” or “block,” as used herein, refers to logic embodied in hardware or firmware, or to a collection of software instructions. A module, a unit, or a block described herein may be implemented as software and/or hardware and may be stored in any type of non-transitory computer-readable medium or another storage device. In some embodiments, a software module/unit/block may be compiled and linked into an executable program. It will be appreciated that software modules can be callable from other modules/units/blocks or from themselves, and/or may be invoked in response to detected events or interrupts. Software modules/units/blocks configured for execution on computing devices may be provided on a computer-readable medium, such as a compact disc, a digital video disc, a flash drive, a magnetic disc, or any other tangible medium, or as a digital download (and can be originally stored in a compressed or installable format that needs installation, decompression, or decryption prior to execution). Such software code may be stored, partially or fully, on a storage device of the executing computing device, for execution by the computing device. Software instructions may be embedded in firmware, such as an EPROM. It will be further appreciated that hardware modules/units/blocks may be included in connected logic components, such as gates and flip-flops, and/or can be included of programmable units, such as programmable gate arrays or processors. The modules/units/blocks or computing device functionality described herein may be implemented as software modules/units/blocks, but may be represented in hardware or firmware. In general, the modules/units/blocks described herein refer to logical modules/units/blocks that may be combined with other modules/units/blocks or divided into sub-modules/sub-units/sub-blocks despite their physical organization or storage. The description may be applicable to a system, an engine, or a portion thereof.

It will be understood that when a unit, engine, module, or block is referred to as being “on,” “connected to,” or “coupled to,” another unit, engine, module, or block, it may be directly on, connected or coupled to, or communicate with the other unit, engine, module, or block, or an intervening unit, engine, module, or block may be present, unless the context clearly indicates otherwise. As used herein, the term “and/or” includes any and all combinations of one or more of the associated listed items. The term “pixel” and “voxel” in the present disclosure are used interchangeably to refer to an element of an image. An anatomical structure shown in an image of a subject (e.g., a patient) may correspond to an actual anatomical structure existing in or on the subject's body.

These and other features, and characteristics of the present disclosure, as well as the methods of operation and functions of the related elements of structure and the combination of parts and economies of manufacture, may become more apparent upon consideration of the following description with reference to the accompanying drawings, all of which form a part of this disclosure. It is to be expressly understood, however, that the drawings are for the purpose of illustration and description only and are not intended to limit the scope of the present disclosure. It is understood that the drawings are not to scale.

In the present disclosure, a representation of a subject (e.g., an object, a patient, or a portion thereof) in an image may be referred to as “subject” for brevity. For instance, a representation of an organ, tissue (e.g., a heart, a liver, a lung), or an scanning region in an image may be referred to as the organ, tissue, or scanning region, for brevity. Further, an image including a representation of a subject, or a portion thereof, may be referred to as an image of the subject, or a portion thereof, or an image including the subject, or a portion thereof, for brevity. Still further, an operation performed on a representation of a subject, or a portion thereof, in an image may be referred to as an operation performed on the subject, or a portion thereof, for brevity. For instance, a segmentation of a portion of an image including a representation of an scanning region from the image may be referred to as a segmentation of the scanning region for brevity.

A movable medical device can be moved to multiple examination rooms to complete multiple medical scans. Each time the movable medical device is moved to an examination room, a user (e.g., a technologist) needs to manually select a scan protocol to be used in the medical scan from a large number of preset scan protocols according to a patient's needs. The efficiency of the determination of the scan protocol is low and the workload of the user is heavy.

An aspect of the present disclosure relates to medical systems and methods for automatically determining a scan protocol for a movable medical device. The systems may obtain first information relating to an examination room where the movable medical device is located. The systems may also determine at least one of environment information of the examination room or subject information relating to one or more subjects in the examination room. The one or more subjects may include a target subject to be scanned. Further, the systems may determine a scan protocol of the movable medical device to be used in a medical scan of the target subject based on the at least one of the environment information or the subject information. Compared with the conventional approach, the systems and methods of the present disclosure is more efficient and accurate by, e.g., reducing the workload of a user, the time needed for determining the scan protocol, and cross-user variations.

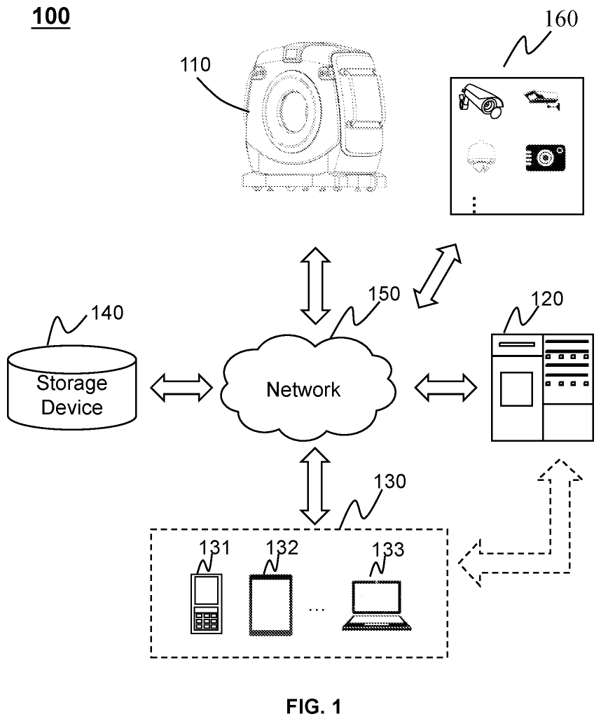

FIG. 1 is a schematic diagram illustrating an exemplary medical system 100 according to some embodiments of the present disclosure. As shown in FIG. 1, the medical system 100 may include a movable medical device 110, a processing device 120, one or more terminals 130, a storage device 140, a network 150, and an image acquisition device 160. In some embodiments, the movable medical device 110, the processing device 120, the terminal(s) 130, and/or the storage device 140 may be connected to and/or communicate with each other via a wireless connection (e.g., the network 150), a wired connection, or a combination thereof. The connection between the components of the medical system 100 may be variable. Merely by way of example, the movable medical device 110 may be connected to the processing device 120 through the network 150, as illustrated in FIG. 1. As another example, the movable medical device 110 may be connected to the processing device 120 directly. As a further example, the terminal(s) 130 may be connected to the processing device 120 through the network 150 or directly. As a further example, the image acquisition device 160 may be connected to the processing device 120 through the network 150 or directly.

The movable medical device 110 may be a non-invasive biomedical movable medical imaging device for disease diagnostic or research purposes. The movable medical device 110 can move to different positions (e.g., different examination rooms or different locations within an examination room) to perform different scans. The movable medical device 110 may be configured to scan a subject (or a part of the subject) to acquire medical image data associated with the subject. The medial image data relating to the subject may be used for generating an anatomical image (e.g., a CT image, an MRI image) of the subject. The anatomical image may illustrate an internal structure of the subject. As used herein, the subject may include a biological subject and/or a non-biological subject. For example, the subject may be a human being, an animal, or a portion thereof. In some embodiments, the subject may be a patient (or a portion thereof).

The movable medical device 110 may include an X-ray scanner, a computed tomography (CT) scanner, a magnetic resonance imaging (MRI) scanner, an ultrasonography scanner, a single photon emission computed tomography (SPECT) scanner, a positron emission tomography (PET) scanner, or the like, or any combination thereof. It should be noted that the movable medical device 110 described below is merely provided for illustration purposes, and not intended to limit the scope of the present disclosure.

For illustration purposes, a movable CT scanner is described hereinafter. The movable CT scanner may include a moving mechanism, a gantry, a scanning device, a control device, a processor, or the like, or any combination thereof. The moving mechanism may be configured to move the movable CT scanner. For example, the movable CT scanner may be moved to different examination rooms or different locations within an examination room via the moving mechanism. In some embodiments, the moving mechanism may include a sliding rail, a wheel, a mechanical leg, a motor, or the like. In some embodiments, via the moving mechanism, the movable CT device can move to a target position automatically or manually driven by a user. The gantry may be connected to the moving mechanism. In some embodiments, the moving mechanism may be installed under the gantry, so that the gantry can be moved along with the movement of the moving mechanism. The scanning device may be installed on the gantry. The scanning device may be configured to scan a subject. The scanning device may include an X-ray tube, a detector, or the like. The control device may be configured to control the movement of the scanning device. In some embodiments, the control device may control the movement of the scanning device by controlling the movement of the moving mechanism. In some embodiments, the control device may control the movement of the gantry to control the movement of the scanning device. In some embodiments, the scanning device may be movable with respect to the gantry, and the control device may control the movement of the scanning device on the gantry.

The processing device 120 may process data and/or information obtained from the movable medical device 110, the terminal(s) 130, the storage device 140, or other components of the medical system 100. For example, the processing device 120 may obtain first information relating to an examination room where the movable medical device 110 is located. The processing device 120 may also determine at least one of environment information of the examination room or subject information relating to one or more subjects in the examination room based on the first information. The one or more subjects may include a target subject to be scanned. Further, the processing device 120 may determine a scan protocol to be used in a medical scan of the target subject based on the at least one of the environment information or the subject information.

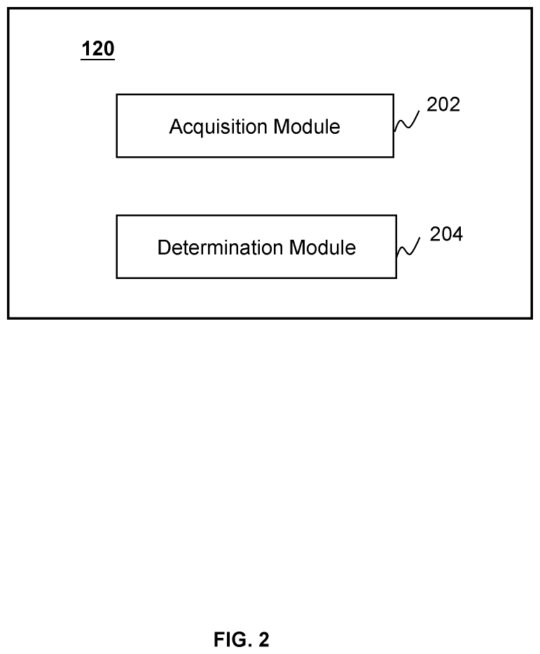

In some embodiments, the processing device 120 (e.g., one or more modules illustrated in FIG. 2) may execute instructions and may accordingly be directed to perform one or more processes (e.g., process 300, 400, and 500) described in the present disclosure. For example, each of the one or more processes may be stored in a storage device (e.g., the storage device 140) as a form of instructions, and invoked and/or executed by the processing device 120.

In some embodiments, the processing device 120 may be a single server or a server group. In some embodiments, the processing device 120 may be local to or remote from the medical system 100. Merely for illustration, only one processing device 120 is described in the medical system 100. However, it should be noted that the medical system 100 in the present disclosure may also include multiple processing devices. Thus, operations and/or method steps that are performed by one processing device 120 as described in the present disclosure may also be jointly or separately performed by the multiple processing devices. For example, if in the present disclosure the processing device 120 of the medical system 100 executes both process A and process B, it should be understood that the process A and the process B may also be performed by two or more different processing devices jointly or separately in the medical system 100 (e.g., a first processing device executes process A and a second processing device executes process B, or the first and second processing devices jointly execute processes A and B).

The terminal(s) 130 may be connected to and/or communicate with the movable medical device 110, the processing device 120, and/or the storage device 140. For example, the terminal(s) 130 may receive a user instruction to determine the scan protocol to be used in the medical scan. As another example, the terminal(s) 130 may display the scan protocol. In some embodiments, the terminal(s) 130 may include a mobile device 131, a tablet computer 132, a laptop computer 133, or the like, or any combination thereof. In some embodiments, the terminal(s) 130 may be part of the processing device 120.

The storage device 140 may store data, instructions, and/or any other information. In some embodiments, the storage device 140 may store data obtained from the processing device 120, the terminal(s) 130, and/or the movable medical device 110. For example, the storage device 140 may store image data collected by the movable medical device 110. As another example, the storage device 130 may store multiple scan protocols used in multiple medical scans. In some embodiments, the storage device 140 may store data and/or instructions that the processing device 120 may execute or use to perform exemplary methods described in the present disclosure.

In some embodiments, the storage device 140 may include a mass storage device, a removable storage device, a volatile read-and-write memory, a read-only memory (ROM), or the like, or any combination thereof. Exemplary mass storage devices may include a magnetic disk, an optical disk, a solid-state drive, etc. In some embodiments, the storage device 150 may be implemented on a cloud platform as described elsewhere in the disclosure.

The network 150 may include any suitable network that can facilitate the exchange of information and/or data for the medical system 100. In some embodiments, one or more components of the medical system 100 (e.g., the movable medical device 110, the processing device 120, the storage device 140, the terminal(s) 130) may communicate information and/or data with one or more other components of the medical system 100 via the network 150. For example, the processing device 120 may obtain the first information relating to an examination room where the movable medical device 110 is located via the network 150.

The image acquisition device 160 may be configured to capture optical image data of subjects located in a field of view of the image acquisition device 160, which may illustrate an external surface of the subjects. For example, the image acquisition device 160 may be configured to capture one or more optical images relating to an examination room where the movable medical device 110 is located. The image acquisition device 160 may be and/or include any suitable device that is capable of capturing optical image data. For example, the image acquisition device 160 may include a camera (e.g., a digital camera, an analog camera, etc.), a red-green-blue (RGB) sensor, an RGB-depth (RGB-D) sensor, or another device that can capture color image data. As another example, the image acquisition device 160 may be used to acquire point-cloud data. The point-cloud data may include a plurality of data points, each of which may represent a physical point on a surface of a subject and can be described using one or more feature values of the physical point (e.g., feature values relating to the position and/or the composition of the physical point). Exemplary image acquisition devices 160 capable of acquiring point-cloud data may include a 3D scanner, such as a 3D laser imaging device, a structured light scanner (e.g., a structured light laser scanner). As yet another example, the image acquisition device 160 may be used to acquire depth image data. The depth image data may refer to image data that includes depth information of each physical point on the body surface of the subject, such as a distance from each physical point to a specific point (e.g., an optical center of the image acquisition device 160). The depth image data may be captured by a range sensing device, e.g., a structured light scanner, a time-of-flight (TOF) device, a stereo triangulation camera, a sheet of light triangulation device, an interferometry device, a coded aperture device, a stereo matching device, or the like, or any combination thereof.

In some embodiments, the image acquisition device 160 may be a device independent from the movable medical device 110. For example, the image acquisition device 160 may be a camera mounted on the ceiling in an examination room where the movable medical device 110 is located or out of the examination room. Alternatively, the image acquisition device 160 may be integrated into or mounted on the movable medical device 110 (e.g., the gantry).

In some embodiments, a medical scan procedure disclosed in the present application may include a plurality of operations. Different operations may be performed based on a same set of image data or different sets of image data captured by one or more image capturing devices 160. For example, the determination of the environment information as described in operation 320, and the determination of a region of interest (scanning region) of the subject that needs to receive the medical scan as described in operation 420, and the determination of a position of the movable medical device relative to the subject may be performed based on a same set of image data or different sets of image data. The different sets of image data of the subject may be captured by different image capturing devices 160 or a same image capturing device 160 at different times.

It should be noted that the above description of the medical system 100 is intended to be illustrative, and not to limit the scope of the present disclosure. Many alternatives, modifications, and variations will be apparent to those skilled in the art. The features, structures, methods, and other characteristics of the exemplary embodiments described herein may be combined in various ways to obtain additional and/or alternative exemplary embodiments. For example, the medical system 100 may include one or more additional components. Additionally or alternatively, one or more components of the medical system 100 described above may be omitted. As another example, two or more components of the medical system 100 may be integrated into a single component.

FIG. 2 is a block diagram illustrating exemplary processing device 120 according to some embodiments of the present disclosure.

As shown in FIG. 2, the processing device 120 may include an acquisition module 202 and a determination module 204.

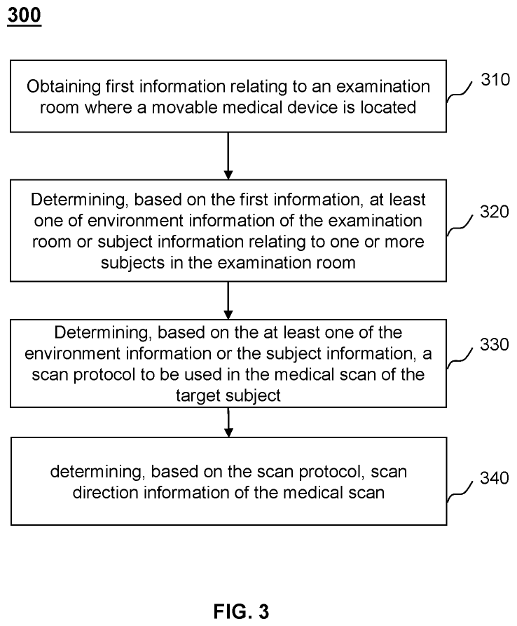

The acquisition module 202 may be configured to obtain information relating to the medical system 100. For example, the acquisition module 202 may obtain first information relating to an examination room where a movable medical device is located. More descriptions regarding the obtaining of the first information may be found elsewhere in the present disclosure. See, e.g., operation 310 in FIG. 3, and relevant descriptions thereof.

In some embodiments, the determination module 204 may be configured to determine at least one of environment information of the examination room or subject information relating to one or more subjects in the examination room based on the first information. The one or more subjects may include a target subject to be scanned. More descriptions regarding the determination of the environment information or the subject information may be found elsewhere in the present disclosure. See, e.g., operation 320 in FIG. 3, and relevant descriptions thereof.

In some embodiments, the determination module 204 may be configured to determine a scan protocol to be used in a medical scan of the target subject based on the at least one of the environment information or the subject information. More descriptions regarding the determination of the scan protocol may be found elsewhere in the present disclosure. See, e.g., operation 330 in FIG. 3, and relevant descriptions thereof.

In some embodiments, the determination module 204 may be configured to determine scan direction information of the medical scan based on the scan protocol. The scan direction information of the medical scan may include moving directions, positions, etc., of one or more components of the medical system 100. For example, the movable medical device is a movable CT scanner, the scan direction information may include a moving direction of a table for supporting the target subject, a rotation direction of a radiation resource that performs the medical scan, or an angle between a rotation plane of the radiation resource and a surface of the table for bearing the target subject, or the like, or any combination thereof. More descriptions regarding the determination of the scan direction information may be found elsewhere in the present disclosure. See, e.g., operation 340 in FIG. 3, and relevant descriptions thereof.

It should be noted that the above description is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. In some embodiments, any one of the modules may be divided into two or more units. For instance, the acquisition module 202 may be divided into two units configured to acquire different data. In some embodiments, the processing device 120 may include one or more additional modules, such as a storage module (not shown) for storing data.

FIG. 3 is a flowchart illustrating an exemplary process 300 for determining a scan protocol to be used in a medical scan of a target subject according to some embodiments of the present disclosure.

In 310, the processing device 120 (e.g., the acquisition module 202) may obtain first information relating to an examination room where a movable medical device is located.

The movable medical device may be configured to perform the medical scan of the target subject. The movable medical device may be similar to the movable medical device 110 as described in connection with FIG. 1. The examination room refers to a room for performing the medical scan on the target subject. The examination room may include a medical imaging room, an operating room, or the like. In some embodiments, the examination room may be used to perform a scan of a specific type (e.g., a scan on a specific part (e.g., a lung of the target subject)), and a same scan protocol may be used for each scan in the examination room. In some embodiments, the examination room may be used to perform examinations of multiple types (e.g., scans on a plurality of parts), and different scan protocols may be used for different examinations. Exemplary operating rooms may include a hybrid operating room, a general operating room, an orthopedic operating room, an obstetrical operating room, a male surgical operating room, a cardiothoracic operating room, a urological operating room, a burn operating room, and an ENT operating room, or the like.

In some embodiments, the examination room may include one or more tables for supporting the target subject. In some embodiments, the movable medical device may automatically navigate to the vicinity of the table where the target subject is located. In some embodiments, the movable medical device may be manually moved to the vicinity of the table where the target subject is located.

In some embodiments, the first information may include image information of the examination room. The image information may include an optical image (also referred to as a first optical image), point-cloud data, etc., captured by the image acquisition device 160. In some embodiments, the image acquisition device 160 may capture the image information before or after the movable medical device enters the examination room. The processing device 120 may obtain the image information from the image acquisition device 160.

In some embodiments, the first information may include position information of the movable medical device in a building where the examination room is located. The position information of the movable medical device may be collected using an indoor positioning technology. Exemplary indoor positioning technologies may include a Wi-Fi technology, a Bluetooth technology, an infrared technology, or the like, or any combination thereof. In some embodiments, the position information of the movable medical device may be collected after the movable medical device is moved into the examination room, therefore the position information of the movable medical device may be regarded as position information of the examination room in the building.

In some embodiments, the first information may be stored in a storage device (e.g., the storage device 140), and the processing device 120 may retrieve the first information from the storage device.

In 320, the processing device 120 (e.g., determination module 204) may determine, based on the first information, at least one of environment information of the examination room or subject information relating to one or more subjects in the examination room.

In some embodiments, the one or more subjects may include the target subject to be scanned and one or more other subjects who participate the medical scan. For example, the one or more other subjects may include a doctor, an assistant, a technologist, or the like, or any combination thereof.

In some embodiments, the subject information relating to one or more subjects may include identity information of the one or more subjects collected by an identification device disposed on the movable medical device 110. The identification device may include an image recognition device (e.g., a camera), a fingerprint recognition device, a voiceprint recognition device (e.g., a microphone), or the like, or any combination thereof. The identify information may include facial information, fingerprint information, voice information, or any information that can be used to determine the identity of the one or more subjects. For example, the image recognition device may collect the identity information of the one or more subjects by performing a face recognition on the one or more subjects. In some embodiments, the processing device 120 may obtain the identity information of the one or more subjects from the identification device or determine the identify information of the one or more subjects based on raw data collected by the identification device.

The environment information of the examination room may include a name or a serial number of the examination room, a scene in the examination room, an internal identification of the examination room, or the like. For example, a mark indicating the name and/or the serial number of the examination room may be located on a side or a top of a door of the examination room. The scene in the examination room may include objects located in the examination room (e.g., a shadowless lamp, a monitoring device, etc. in an operating room). The scene in the examination room may also include a spatial layout of objects in the examination room. For example, the spatial layout may include a location of the table for supporting the target subject, a space around the table, locations of objects around the table, locations of other objects in the examination room, or the like. The internal identification of the examination room may include a usage description, announcements, etc., of the examination room.

In some embodiments, the first information may include the first optical image of the examination room, the processing device 120 may determine the environment information of the examination room by analyzing the first optical image. For example, the processing device 120 may identify the name, the serial number, internal identification, etc. of the examination room from the first optical image using a text recognition technology such as an optical character recognition (OCR) technology.

In some embodiments, the first information includes the image information of the examination room (e.g., the first optical image or the point-cloud data), and the processing device 120 may determine the environment information of the examination room using a determination model based on the image information. In some embodiments, the image information may include two-dimensional (2D) image information or 3D image information. Specifically, the image information may be input into the determination model, and the determination model may output the environment information of the examination room. In some embodiments, the determination model may be a trained machine learning model. In some embodiments, the determination model may include a deep learning model, such as a Deep Neural Network (DNN) model, a Convolutional Neural Network (CNN) model, a Recurrent Neural Network (RNN) model, a Feature Pyramid Network (FPN) model, etc. Exemplary CNN models may include a V-Net model, a U-Net model, a Link-Net model, or the like, or any combination thereof.

In some embodiments, the processing device 120 may obtain the determination model from one or more components of the medical system 100 (e.g., the storage device 140, the terminals(s) 130) or an external source via a network (e.g., the network 150). For example, the determination model may be previously trained by a computing device (e.g., the processing device 120), and stored in a storage device (e.g., the storage device 140) of the medical system 100. The processing device 120 may access the storage device and retrieve the determination model. Merely by way of example, the determination model may be generated by training a first preliminary model using a plurality of first training samples. Each first training sample may include sample image information of a sample examination room and sample environment information of the sample examination room. The sample environment information of the sample examination room can be used as a ground truth (also referred to as a label) for model training. In some embodiments, the sample environment information may be determined by a user. Since the determination model may learn the optimal mechanism for determining environment information of an examination room based on a large amount of data, the environment information determined using the determination model may be relatively accurate, thereby in turn improving the accuracy of the scan protocol determined based on the environment information.

In some embodiments, for each of one or more reference examination rooms, the processing device 120 may obtain reference image information of the reference examination room and reference environment information corresponding to the reference examination room. The reference image information may include image information with a same type as the image information. For example, the image information and the reference image information may be both optical images. As another example, the image information and the reference image information may be both point-cloud data. Further, the processing device 120 may generate a comparison result by comparing the image information with the reference image information of each reference examination room. The comparison result may include a similarity between reference image information of each reference examination room and the image information. In some embodiments, a similarity between reference image information of a reference examination room and the image information refers to a similarity between at least a portion of the reference image information and at least a portion of the image information. For example, the similarity between the reference image information and the image information refers to a similarity between one or more reference objects (e.g., a shadowless lamp, a monitoring device, an operation device, etc.) in the reference image information and on or more objects in the image information corresponding to the one or more reference objects. For brevity, a similarity between reference image information of a reference examination room and the image information may be also referred to as a similarity of the reference examination room.

Then, the processing device 120 may determine the environment information of the examination room based on the comparison result and the reference environment information corresponding to each reference examination room. For example, the processing device 120 may determine a reference examination room with a largest similarity among the one or more reference examination rooms, and designate the reference environment information of the reference examination room as the environment information of the examination room. As another example, the processing device 120 may determine one or more candidate examination rooms whose similarities are larger than a similarity threshold among the one or more reference examination rooms. Based on the reference environment information of one of the one or more candidate examination rooms, the environment information of the examination room may be determined.

In some embodiments, the first information includes position information of the movable medical device in the building where the examination room is located, and the processing device 120 may determine a position of the examination room in the building based on the positioning information of the movable medical device. Further, the processing device 120 may determine the environment information of the examination room based on the position of the examination room. For example, a corresponding relationship between each examination room in the building and one or more medical scans that can be performed in the examination room may be previously determined and stored in a storage device (e.g., the storage device 140). The processing device 120 may determine the environment information of the examination room according to the position of the examination room and the corresponding relationship.

In 330, the processing device 120 (e.g., the determination module 204) may determine, based on the at least one of the environment information or the subject information, a scan protocol to be used in the medical scan of the target subject.

In some embodiments, the medical scan may be performed by the movable medical device. The scan protocol may include one or more scanning parameters to be used in the medical scan. One or more components of the movable medical device may be adjusted according to the one or more scanning parameters. For example, if the movable medical device needs to perform a CT scan, the one or more scanning parameters may include a scanning mode, an X-rays dose, a focus, a gain, a tube voltage, an effective tube current, or the like, or any combination thereof. The scan mode may include an axial scan, a helical scan, etc. In some embodiments, different portions of the target subject may correspond to different scan protocols. Optionally, different scan protocols may be performed on the same portion of different target subjects depending on the conditions of the target subjects. Exemplary scan protocols may include a spine axial scan protocol, an abdominal helical scan protocol, an inner ear high fraction axial scan protocol, or the like.

In some embodiments, the processing device 120 may determine the scan protocol based on the environment information of the examination room. In some embodiments, one or more preset scan protocols corresponding to different examination rooms may be previously determined and stored in a storage device (e.g., the storage device 140). The processing device 120 may retrieve one or more preset scan protocols corresponding to the examination room from the storage device according to the environment information of the examination room, and determine the scan protocol from the one or more preset scan protocols corresponding to the examination room. If there is only one preset scan protocol corresponding to the examination room, the processing device 120 may directly determine the preset scan protocol as the scan protocol. For example, the examination room is a physical examination room for chest scan, there may be only one preset scan protocol corresponding to the physical examination room, and the processing device 120 may directly determine the preset scan protocol as the scan protocol. As another example, the examination room is an operating room, there is only one operation needs to performed in the operation room at the current time. A user may previously determine a preset scan protocol to be used in the operation, and the processing device 120 may directly the preset scan protocol as the scan protocol.

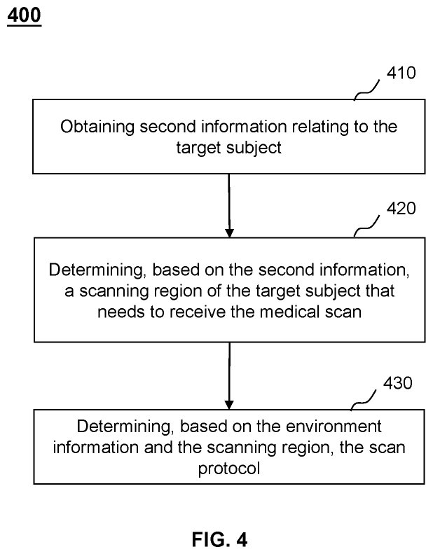

If there are multiple preset scan protocols corresponding to the examination room, the processing device 120 may determine the scan protocol from the multiple preset scan protocols. For example, the multiple preset scan protocols may correspond to different regions in the examination room. The processing device 120 may determine a region of the examination room where the movable medical device is located. Further, the processing device 120 may determine one preset scan protocol from the multiple preset scan protocols as the scan protocol according to the region of the examination room where the movable medical device is located. As another example, the multiple preset scan protocols may correspond to different body parts. The processing device 120 may obtain second information relating to the target subject. Further, the processing device 120 may determine a scanning region of the target subject that needs to receive the medical scan based on the second information. Then, the processing device 120 may determine the scan protocol based on the environment information and the scanning region. More descriptions regarding the determination of the scan protocol based on the environment information and the scanning region may be found elsewhere in the present disclosure (e.g., FIG. 4 and the descriptions thereof).

In some embodiments, if there is no preset scan protocol corresponding to the examination room, the processing device 120 obtain a plurality of reference scan protocols corresponding to the scanning region of the target subject from a database for storing scan protocols, and select one reference scan protocol from the plurality of reference scan protocols as the scan protocol.

In some embodiments, the processing device 120 may determine the scan protocol based on the subject information relating to one or more subjects. In some embodiments, the subject information relating to one or more subjects may include the identity information of the one or more subjects, the processing device 120 may determine the scan protocol based on the identity information of the one or more subjects by retrieving a medical database. The medical database may store identify information of multiple subjects and scan protocols of each subject. For example, after a doctor diagnoses the target subject, the doctor may send diagnosis information to the medical database. The diagnosis information may include the identity information of the target subject, the identity information of the doctor, information relating to the medical scan to be performed on the target subject (e.g., a scan protocol to be used in the medical scan). The processing device 120 may determine the scan protocol based on the identity information of the one or more subjects by retrieving the medical database.

In some embodiments, after the scan protocol is determined, the processing device 120 may proceed to one or more other operations. For example, the processing device 120 may determine one or more scan parameters of the movable medical device used in medical scan based on the scan protocol, and further direct the movable medical device to perform the medical scan based on the one or more scan parameters. Optionally, the one or more parameters may be confirmed or adjusted by a user before the movable medical device is directed to perform the medical scan.

As another example, after the medical scan is performed on the target subject, the processing device 120 obtain scan data collected via the medical scan, and further reconstruct a medical image of the target subject based on the scan data. In some embodiments, the scan data may be preprocessed, and the processing device 120 may further reconstruct the medical image of the target subject based on the preprocessed scan data. As still another example, the processing device 120 may determine one or more objects that need to be displayed on a user interface based on an optical image of the examination room captured by the image acquisition device 160 (e.g., the first optical image). For example, when a surgery of the target subject is performed, the processing device 120 may determine one or more surgical devices that need to be displayed on the user interface (e.g., an interventional needle used to determine a portion of the target subject that needs to receive the surgery). As still another example, the processing device 120 may perform operation 340 to determine scan direction information of the medical scan.

In some occasions, a plurality of medical scans (e.g., a scan of a spine, a scan of the brain, a scan of the chest, etc.) to be performed by the movable medical device may be performed in different regions in the examination room. Each time one of plurality of medical scans needs to be performed (which is referred to as a target medical scan), the processing device 120 may determine a target scan protocol to be used in the target medical scan. In some embodiments, the processing device 120 may determine a region of the examination room where the movable medical device is located. Further, the processing device 120 may determine the target medical scan from the plurality of medical scans according to the region of the examination room where the movable medical device is located. Then, the processing device 120 may determine the target scan protocol to be used in the target medical scan in a similar manner as determination of the scan protocol to be used in the medical scan of the target subject based on the environment information or the subject information. For example, the processing device 120 may obtain one or more preset scan protocols corresponding to the region of the examination room where the movable medical device is located. If there is only one preset scan protocol corresponding to the region, the processing device 120 may directly determine the preset scan protocol as the target scan protocol. If there are multiple preset scan protocols corresponding to the region, the processing device 120 may determine the target scan protocol from the multiple preset scan protocols. For example, the multiple preset scan protocols may correspond to different body parts. The processing device 120 may obtain second information relating to a subject that needs to receives the target scan. Further, the processing device 120 may determine a scanning region of the subject that needs to receive the target medical scan based on the second information. Then, the processing device 120 may determine the target scan protocol based on the environment information and the scanning region. In some embodiments, if there is no preset scan protocol corresponding to the region, the processing device 120 obtain a plurality of reference scan protocols corresponding to the scanning region of the subject from a database for storing scan protocols, and select one reference scan protocol from the plurality of reference scan protocols as the target scan protocol.

As described elsewhere in the present disclosure, conventionally, before a medical scan is started, a user (e.g., a technologist) needs to select a scan protocol to be used in the medical scan from a large number of preset scan protocols according to a patient's needs. The efficiency of the determination of the scan protocol is low and the workload of the user is heavy.

According to some embodiments of the present disclosure, the scan protocol may be automatically determined based on at least the first information of the examination room (e.g., image information and/or position information of the examination room). Compared with the conventional approach, the systems and methods of the present disclosure is more efficient and accurate by, e.g., reducing the workload of a user, and the time needed for determining the scan protocol, and cross-user variations.

In 340, the processing device 120 (e.g., the determination module 204) may determine, based on the scan protocol, scan direction information of the medical scan.

The scan direction information of the medical scan may include moving directions, positions, etc., of one or more components of the medical system 100. For example, the movable medical device is a movable CT scanner, the scan direction information may include a moving direction of a table for supporting the target subject, a rotation direction of a radiation resource that performs the medical scan, an angle between a rotation plane of the radiation resource and a surface of the table for bearing the target subject, or the like, or any combination thereof.

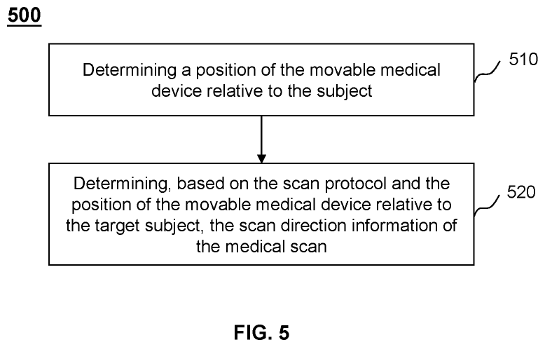

In some embodiments, the processing device 120 may determine a position of the movable medical device relative to the target subject. Further, the processing device 120 may determine the scan direction information of the medical scan based on the scan protocol and the position of the movable medical device relative to the target subject. More descriptions regarding the determination of the scanning direction information may be found elsewhere in the present disclosure (e.g., FIG. 5 and relevant descriptions thereof).

It should be noted that the above description regarding the process 300 is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. In some embodiments, the process 300 may be accomplished with one or more additional operations not described and/or without one or more of the operations discussed above. For example, the process 300 may include an additional operation to transmit the scan protocol to a terminal device (e.g., a terminal device 130 of a doctor) for display. As another example, operation 340 may be omitted.

FIG. 4 is a flowchart illustrating an exemplary process 400 for determine a scan protocol to be used in a medical scan of a target subject according to some embodiments of the present disclosure. In some embodiments, one or more operations of the process 400 may be performed to achieve at least part of operation 330 as described in connection with FIG. 3.

In 410, the processing device 120 (e.g., the determination module 204) may obtain second information relating to the target subject.

The second information include information relating to a scanning region of the target subject. The scanning region may include the abdomen, the head, the chest, limbs, etc., or any portion of the target subject that needs to receive the medical scan.

In some embodiments, the second information may include image information of the target subject. For example, the second information may include a second optical image of the target subject (or a portion of the target subject) captured by the image acquisition device 160. The second optical image may include a portion of the target subject covered by a surgical drape and another portion that is not covered by the surgical drape (also referred to as an uncovered portion). The uncovered portion may include the scanning region of the target subject, a marker enclosed in the scanning region, a surgical instrument (e.g., an interventional needle) placed near the scanning region, or the like. In some embodiments, the processing device 120 may obtain the second optical image of the target subject from the image acquisition device 160. In some embodiments, the second optical image may be the same as the first optical image.

In some embodiments, the second information may include a medical image of the target subject (or a portion of the target subject). The medical image may include the scanning region. The medical image of the target subject may be generated by a biomedical scan technique as described elsewhere in this disclosure. For example, the medical image may include a DR image, an MR image, a PET image, a CT image, a PET-CT image, a PET-MR image, an ultrasound image, etc. In some embodiments, the processing device 120 may obtain the medical image of the target subject from a device for generating the medical image.

In some embodiments, the second information may include identity information of the target subject collected by an identification device disposed on the movable medical device (e.g., the identification device described in operation 320). In some embodiments, the processing device 120 may obtain the identity information of the target subject from the identification device.

It should be noted that the above descriptions regarding the second information are merely provided for illustration purposes, and not intended to be limiting. For example, the second information may include a diagnosis result, a treatment record, or any other information that can be used to determine the scanning region.

In some embodiments, the second information may be previously generated and stored in a storage device (e.g., the storage device 140 or an external source). The processing device 120 may retrieve the second information from the storage device.

In 420, the processing device 120 (e.g., the determination module 204) may determine, based on the second information, the scanning region of the target subject that needs to receive the medical scan.

In some embodiments, the second information may include the second optical image of the target subject. The processing device 120 may directly identify the scanning region from the second optical image. For example, if the uncovered portion displayed in the second optical image is the head, the processing device 120 may determine the head as the scanning region. In some embodiments, the identification of the uncovered portion may be performed using an image identification algorithm, such as a trained image identification model.

In some embodiments, the second information may include an image of the target subject (e.g., the second optical image or a medial image of the target subject), and the scanning region may be determined by processing the image using a region determination model. Specifically, the image may be input into the region determination model, and the region determination model may output information relating to the scanning region (e.g., a segmentation mask, position information of the scanning region, etc.). The region determination model may be a trained machine learning model. In some embodiments, the region determination model may include any model as described elsewhere in the present disclosure.

In some embodiments, the processing device 120 may obtain the region determination model in a similar manner as how the determination model is obtained described in operation 320. In some embodiments, the region determination model may be generated by training a second preliminary model using a plurality of second training samples. Each second training sample may include a sample image relating to a sample target subject and a sample scanning region of the sample target subject. In some embodiments, types of the sample images for training the region determination model and the image of the target subject may be the same. For example, the sample images and the image of the target subject may be optical images. Alternatively, the sample images and the image of the target subject may be medical images. The sample scanning region of the sample target subject can be used as a ground truth (also referred to as a label) for model training. In some embodiments, the sample medical image relating to the sample target subject may be generated by a biomedical scan technique as described elsewhere in this disclosure. In some embodiments, the sample scanning region may be a region of the sample target subject that is scanned. Since the region determination model may learn the optimal mechanism for scanning region determination based on a large amount of data, the scanning region determined using the determination model may be relatively accurate, thereby in turn improving the accuracy of the scan protocol determined based on the environment information and the scanning region.

In some embodiments, the second information may include the identity information of the target subject, and the processing device 120 may determine the scanning region based on the identity information of the target subject by retrieving a medical database. For example, after a doctor diagnoses the target subject, the doctor may send diagnosis information to the medical database. The diagnosis information may include the identity information of the target subject, information relating to medical scan that includes the scanning region that needs to receive the medical scan. The processing device 120 may determine the scanning region using the identity information of the target subject by retrieving the medical database.

In 430, the processing device 120 (e.g., the determination module 204) may determine, based on the environment information and the scanning region, the scan protocol.

In some embodiments, the processing device 120 may obtain one or more candidate scan protocols based on the environment information. The one or more candidate scan protocols may include one or more preset scan protocols corresponding to the examination room and/or one or more historical scan protocols of one or more historical medical scans corresponding to the examination room. As used herein, a scan protocol corresponding to the examination room refers to a scan protocol that can be used in a medical scan performed in the examination room. In some embodiments, as described in operation 330, multiple preset scan protocols corresponding to different examination rooms may be previously generated and stored in a storage device (e.g., the storage device 140). The processing device 120 may retrieve the one or more preset scan protocols corresponding to the examination room from the storage device according to the environment information of the examination room, and designate the one or more preset scan protocols corresponding to the examination room as one or more candidate scan protocols. In some embodiments, the processing device 120 may obtain one or more historical medical scans that have been performed by the movable medical device in the examination room. The processing device 120 may designate one or more historical scan protocols of the one or more historical medical scans as one or more candidate scan protocols.

Further, the processing device 120 may select the scan protocol based on the scanning region from the one or more candidate scan protocols. For example, the one or more candidate scan protocols may correspond to different body parts. The processing device 120 may select one candidate scan protocol corresponding to a body part including the scanning region as the scan protocol. In some embodiments, there are a plurality of candidate scan protocols corresponding to the body part including the scanning region as the scan protocol, the processing device 120 may send the plurality of candidate scan protocols to a user, and the user may determine one of the plurality of candidate scan protocols as the scan protocol. Alternatively, the processing device 120 may select one candidate scan protocol as the scan protocol randomly or based on feature information of the target subject and feature information of historical target subjects who receive historical medical scans performed based on the candidate scan protocols.

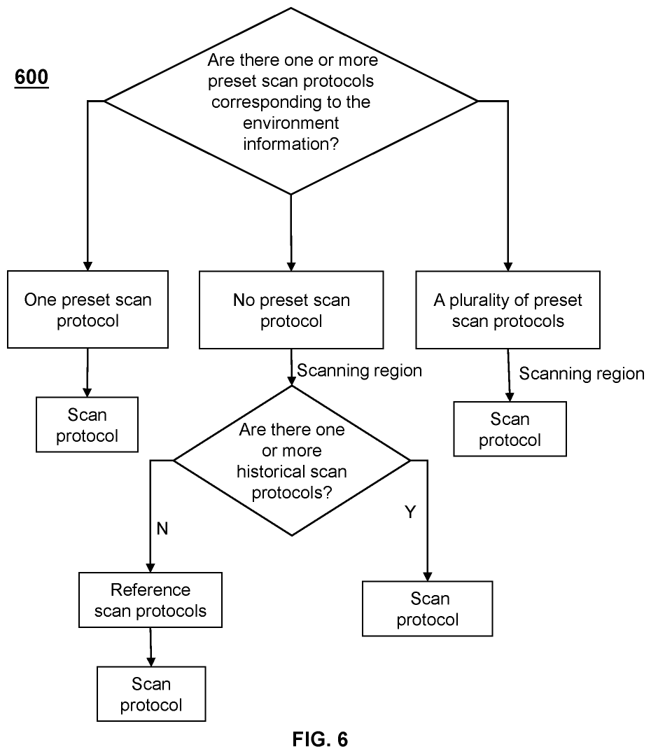

For illustration purposes, FIG. 6 provides a schematic diagram illustrating an exemplary process for scan protocol determination according to some embodiments of the present disclosure. As illustrated in FIG. 6, the processing device 120 may determine whether there are the one or more preset scan protocols corresponding to the examination room. In response to determining that there is only one preset scan protocol corresponding to the examination room, the processing device 120 may directly designate the preset scan protocol as the scan protocol. In response to determining that there are a plurality of preset scan protocols, the processing device 120 may determine the scan protocol based on the plurality of preset scan protocols and the scanning region. For example, the plurality of preset scan protocols may correspond to different body parts. The processing device 120 may select one preset scan protocol corresponding to a body part including the scanning region as the scan protocol. In response to determining that there is no preset scan protocol, the processing device 120 may determine the scan protocol based on the scanning region. For example, the processing device 120 may determine whether there are one or more historical medical scans that are performed on a part including the scanning region via the movable medical device in the examination room. In response to determining that there are one or more historical medical scans that are performed on a body part including the scanning region via the movable medical device in the examination room, the processing device 120 may obtain one or more historical scan protocols of the one or more historical medical scans, and select one of the one or more historical scan protocols as the scan protocol. In response to determining that there is no historical medical scan, the processing device 120 may obtain a plurality of reference scan protocols corresponding to the scanning region of the target subject from a database for storing scan protocols. The processing device 120 may send the plurality of reference scan protocols to a user, and the user may select one of the plurality of reference scan protocols as the scan protocol. Alternatively, the processing device 120 may select one reference scan protocol as the scan protocol randomly or based on feature information of the target subject and feature information of historical target subjects who receive historical medical scans performed based on the reference scan protocols.

FIG. 5 is a flowchart illustrating an exemplary process 500 for determining scan direction information of a medical scan of a target subject according to some embodiments of the present disclosure. In some embodiments, one or more operations of the process 500 may be performed to achieve at least part of operation 340 as described in connection with FIG. 3.

As described in operation 340, the scan direction information of the medical scan may include moving directions, positions, etc., of one or more components of the medical system 100 in the medical scan. For example, the medical scan is a CT scan, the scan direction information may include a moving direction of a table for supporting the target subject, a rotation direction of a radiation resource that performs the medical scan, an angle between a rotation plane of the radiation resource and a surface of the table for bearing the target subject, or the like, or any combination thereof. For illustration purposes, a CT scan is described hereinafter as an example of the medical scan.

In 510, the processing device 120 (e.g., the determination module 204) may determine a position of the movable medical device relative to the target subject.

As used herein, the position of the movable medical device relative to the target subject may indicate which side of the movable medical device the target subject a target portion (e.g., the head, the feet) of the target subject is on and which side of the movable medical device the scanning region is on. For example, the CT scan may be performed by a movable CT scanner with a C-shape gantry for bearing the radiation resource of the movable CT scanner (e.g., a gantry shown in FIG. 1). The gantry may include a first side and a second side along a length direction of the table (e.g., a left side and a right side of the gantry as shown in FIG. 1). The position of the movable medical device relative to the subject may indicate that how the target subject is lied on the table, for example, the head of the target subject is located on the first side of the gantry (also referred to as the first side of the movable CT scanner) and the feet of the subject is located on the second side of the gantry (also referred to as the second side of the movable CT scanner) or vice versa. The position of the movable medical device relative to the subject may also indicate a relative positional relationship between the scanning region and the first side and the second side. For example, the position of the movable medical device relative to the subject may also indicate that the scanning region is on the first side. As another example, the position of the movable medical device relative to the subject may also indicate that a portion of the scanning region is on the first side, and another portion of the scanning region is on the second side.

In some embodiments, the processing device 120 may obtain a third optical image captured by the image acquisition device 160. The third optical image may include the movable medical device and the target subject. In some embodiments, the third optical image and the first optical image may be a same optical image. In some embodiments, the third optical image and the second optical image may be a same optical image. The processing device 120 may the position of the movable medical device relative to the subject based on the third optical image. For example, the processing device 120 may determine the position of the movable medical device relative to the subject by analyzing the third optical image using an image recognition technology.

In 520, the processing device 120 (e.g., the determination module 204) may determine, based on the scan protocol and the position of the movable medical device relative to the target subject, the scan direction information of the medical scan.

In some embodiments, the moving direction of the table may be from the first side to the second side or from the second side to the first side. The rotation direction of the radiation resource may include a clockwise or a counterclockwise. The angle between the rotation plane of the radiation resource and the surface of the table may be in a range of 0-180°.

In some embodiments, the processing device 120 may determine a scanning direction of the scanning region (e.g., a direction from the head to the feet, or a direction from the feet to the head) according to the scan protocol. Further, the processing device 120 may determine the scan direction information based on the scanning direction of the scanning region and the position of the movable medical device relative to the target subject.

For example, the processing device 120 may obtain preset scan direction information. The preset scan direction information indicates that the moving direction of the table is from the first side to the second side and the rotation direction of the radiation resource is a clockwise. If the feet and the scanning region of the target subject are on the first side of the movable CT scanner and the scanning direction at of the scanning region is from the head to the feet, during the CT scan, the moving direction of the table may be from the first side to the second side, and the rotation direction of the radiation resource may be the clockwise. The angle (also referred to as a first angle) between the rotation plane of the radiation resource and the surface of the table may be determined based on the third optical image. For example, the processing device 120 may determine the first angle by analyzing the third optical image. If the head and at least a portion of the scanning region of the target subject are on the first side of the movable CT scanner and the scanning direction at of the scanning region is from the head to the feet, the scanning region needs to be the second side by moving the table, during the CT scan, the moving direction of the table may be from the second side to the first side, and the rotation direction of the radiation resource may be the counterclockwise. The angle (also referred to as a second angle) between the rotation plane of the radiation resource and the surface of the table may be determined based on the third optical image. The second angle may be the supplementary angle of the first angle. In some embodiments, the processing device 120 may determine whether the angle between the rotation plane of the radiation resource and the surface of the table satisfies a requirement. If the angle between the rotation plane of the radiation resource and the surface of the table does not satisfy the requirement, the processing device 120 may generate an instruction indicating that the angle between the rotation plane of the radiation resource and the surface of the table needs to be adjusted.

Conventionally, the scan direction information of the movable medical device is set in advance, and the scanning direction information of the mobile medical device is the same when performing any scan. For example, during any CT scan of the movable CT scanner, the table for supporting the target subject can only move from the first side of the movable CT scanner to the second side, the radiation resource that performs the CT scan can only rotate clockwise, and the angle between the rotation plane of the radiation resource and the surface of the table for bearing the target subject can only be a fixed value. Therefore, before each scan is performed, the positions of the movable medical device and the target subject need to be adjusted, therefore the efficiency of the CT scan is low and the workload of the user is heavy.

According to some embodiments of the present disclosure, the scan direction information of the movable CT scanner may be automatically determined based the scan protocol and the position of the movable medical device relative to the target subject, which may obviate the need for a user to manually adjust the positions of the movable medical device and the target subject, thereby reducing the manual workload and improving the scanning efficiency.

It will be apparent to those skilled in the art that various changes and modifications can be made in the present disclosure without departing from the spirit and scope of the disclosure. In this manner, the present disclosure may be intended to include such modifications and variations if the modifications and variations of the present disclosure are within the scope of the appended claims and the equivalents thereof. For example, the operations of the illustrated processes 300-600 are intended to be illustrative. In some embodiments, the processes 300-600 may be accomplished with one or more additional operations not described, and/or without one or more of the operations discussed. Additionally, the order in which the operations of the processes 300-600 and regarding descriptions are not intended to be limiting.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure, and are within the spirit and scope of the exemplary embodiments of this disclosure.

Moreover, certain terminology has been used to describe embodiments of the present disclosure. For example, the terms “one embodiment,” “an embodiment,” and “some embodiments” mean that a particular feature, structure or characteristic described in connection with the embodiment is included in at least one embodiment of the present disclosure. Therefore, it is emphasized and should be appreciated that two or more references to “an embodiment” or “one embodiment” or “an alternative embodiment” in various portions of this specification are not necessarily all referring to the same embodiment. Furthermore, the particular features, structures or characteristics may be combined as suitable in one or more embodiments of the present disclosure.

Further, it will be appreciated by one skilled in the art, aspects of the present disclosure may be illustrated and described herein in any of a number of patentable classes or context including any new and useful process, machine, manufacture, or composition of matter, or any new and useful improvement thereof. Accordingly, aspects of the present disclosure may be implemented entirely hardware, entirely software (including firmware, resident software, micro-code, etc.) or combining software and hardware implementation that may all generally be referred to herein as a “module,” “unit,” “component,” “device,” or “system.” Furthermore, aspects of the present disclosure may take the form of a computer program product embodied in one or more computer readable media having computer readable program code embodied thereon.

A computer readable signal medium may include a propagated data signal with computer readable program code embodied therein, for example, in baseband or as part of a carrier wave. Such a propagated signal may take any of a variety of forms, including electro-magnetic, optical, or the like, or any suitable combination thereof. A computer readable signal medium may be any computer readable medium that is not a computer readable storage medium and that may communicate, propagate, or transport a program for use by or in connection with an instruction execution system, apparatus, or device. Program code embodied on a computer readable signal medium may be transmitted using any appropriate medium, including wireless, wireline, optical fiber cable, RF, or the like, or any suitable combination of the foregoing.

Computer program code for carrying out operations for aspects of the present disclosure may be written in any combination of one or more programming languages, including an subject oriented programming language such as Java, Scala, Smalltalk, Eiffel, JADE, Emerald, C++, C#, VB. NET, Python or the like, conventional procedural programming languages, such as the “C” programming language, Visual Basic, Fortran 2003, Perl, COBOL 2002, PHP, ABAP, dynamic programming languages such as Python, Ruby and Groovy, or other programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider) or in a cloud computing environment or offered as a service such as a Software as a Service (SaaS).

Furthermore, the recited order of processing elements or sequences, or the use of numbers, letters, or other designations therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software only solution, e.g., an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various embodiments. This method of disclosure, however, is not to be interpreted as reflecting an intention that the claimed subject matter requires more features than are expressly recited in each claim. Rather, claim subject matter lie in less than all features of a single foregoing disclosed embodiment.

In some embodiments, the numbers expressing quantities or properties used to describe and claim certain embodiments of the application are to be understood as being modified in some instances by the term “about,” “approximate,” or “substantially.” For example, “about,” “approximate,” or “substantially” may indicate a certain variation (e.g., ±1%, ±5%, ±10%, or ±20%) of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable. In some embodiments, a classification condition used in classification or determination is provided for illustration purposes and modified according to different situations. For example, a classification condition that “a value is greater than the threshold value” may further include or exclude a condition that “the probability value is equal to the threshold value.”

What is claimed is:

1. A medical system, comprising:
at least one storage device including a set of instructions; and
at least one processor in communication with the at least one storage device, wherein when executing the set of instructions, the at least one processor is configured to direct the system to perform operations including:
obtaining first information relating to an examination room where a movable medical device is located;
determining, based on the first information, at least one of environment information of the examination room or subject information relating to one or more subjects in the examination room, the one or more subjects including a target subject to be scanned; and
determining, based on the at least one of the environment information or the subject information, a scan protocol to be used in a medical scan of the target subject.

2. The system of claim 1, wherein the first information includes image information of the examination room, and the environment information of the examination room is determined based on the image information of the examination room using a determination model, the determination model being a trained machine learning model.

3. The system of claim 1, wherein the first information includes image information of the examination room, and the environment information of the examination room is determined based on the image information of the examination room by performing operations including:
for each of one or more reference examination rooms, obtaining reference image information of the reference examination room and reference environment information corresponding to the reference examination room;
generating a comparison result by comparing the image information with the reference image information of each reference examination room;
determining, based on the comparison result and the reference environment information corresponding to each reference examination room, the environment information of the examination room.

4. The system of claim 1, wherein the first information includes position information of the movable medical device in a building where the examination room is located, and the environment information of the examination room is determined based on the position information of the examination room by performing operations including:
determining, based on the position information of the movable medical device, a position of the examination room in the building; and
determining, based on the position of the examination room, the environment information of the examination room.

5. The system of claim 1, wherein the scan protocol to be used in the medical scan of the target subject is determined based on the environment information by:
obtaining one or more one or more preset scan protocols corresponding to the examination room according to the environment information of the examination room; and
determining, from the one or more preset scan protocols, the scan protocol.

6. The system of claim 1, wherein the scan protocol to be used in the medical scan of the target subject is determined based on the environment information by:
obtaining second information relating to the target subject;
determining, based on the second information, a scanning region of the target subject that needs to receive the medical scan; and
determining, based on the environment information and the scanning region, the scan protocol.

7. The system of claim 6, wherein the determining, based on the environment information and the scanning region, the scan protocol comprises:
obtaining one or more candidate scan protocols based on the environment information, the one or more candidate scan protocols including one or more preset scan protocols corresponding to the examination room and/or one or more historical scan protocols of one or more historical medical scans corresponding to the examination room;
selecting, from the one or more candidate scan protocols, the scan protocol based on the scanning region.

8. The system of claim 6, wherein the determining, based on the environment information and the scanning region, the scan protocol comprises:
determining whether there are one or more preset scan protocols corresponding to the examination room;
in response to determining that there are one or more preset scan protocols corresponding to the examination room, determining the scan protocol based on the one or more preset scan protocols; or
in response to determining that there is no preset scan protocol, determining the scan protocol based on the scanning region.

9. The system of claim 6, wherein the second information includes image information of the target subject, and the scanning region is determined by processing the image using a region determination model, the region determination model being a trained machine learning model.

10. The system of claim 6, wherein the second information includes identity information of the target subject collected by an identification device disposed on the movable medical device, and the scanning region is determined based on the identity information of the target subject by retrieving a medical database.

11. The system of claim 1, wherein the subject information relating to one or more subjects includes identity information of the one or more subject collected by an identification device disposed on the movable medical device, and the scan protocol is determined based on the identity information of the one or more subjects by retrieving a medical database.

12. The system of claim 1, wherein the scan protocol to be used in the medical scan of the target subject is determined based on the environment information by:
determining whether there are a plurality of preset scan protocols correspond to different regions in the examination room; and
in response to determining that there are a plurality of preset scan protocols correspond to different regions in the examination room, determining the scan protocol based on a region of the examination room where the movable medical device is located.

13. The system of claim 1, wherein the operations further include:
determining, based on the scan protocol, scan direction information of the medical scan, the scan direction information including at least one of a moving direction of a table for supporting the target subject, a rotation direction of a radiation resource that performs the medical scan, or an angle between a rotation plane of the radiation resource and a surface of the table for bearing the target subject.

14. The system of claim 13, wherein the determining, based on the scan protocol, scan direction information of the medical scan includes:
determining a position of the movable medical device relative to the target subject; and
determining, based on the scan protocol and the position of the movable medical device relative to the target subject, the scan direction information of the medical scan.

15. A method, the method being implemented on a computing device having at least one storage device and at least one processor, the method comprising:
obtaining first information relating to an examination room where a movable medical device is located;
determining, based on the first information, at least one of environment information of the examination room or subject information relating to one or more subjects in the examination room, the one or more subjects including a target subject to be scanned; and
determining, based on the at least one of the environment information or the subject information, a scan protocol to be used in a medical scan of the target subject.

16. The method of claim 15, wherein the first information includes image information of the examination room, and the environment information of the examination room is determined based on the image information of the examination room using a determination model, the determination model being a trained machine learning model.

17. The method of claim 15, wherein the first information includes image information of the examination room, and the environment information of the examination room is determined based on the image information of the examination room by performing operations including:
for each of one or more reference examination rooms, obtaining reference image information of the reference examination room and reference environment information corresponding to the reference examination room;
generating a comparison result by comparing the image information with the reference image information of each reference examination room
determining, based on the comparison result and the reference environment information corresponding to each reference examination room, the environment information of the examination room.

18. The method of claim 15, wherein the scan protocol to be used in the medical scan of the target subject is determined based on the environment information by:
obtaining second information relating to the target subject;
determining, based on the second information, a scanning region of the target subject that needs to receive the medical scan; and
determining, based on the environment information and the scanning region, the scan protocol.

19. The method of claim 15, wherein the method further includes:
determining, based on the scan protocol, scan direction information of the medical scan, the scan direction information including at least one of a moving direction of a table for supporting the target subject, a rotation direction of a radiation resource that performs the medical scan, or an angle between a rotation plane of the radiation resource and a surface of the table for bearing the target subject.

20. A non-transitory computer readable medium, comprising at least one set of instructions, wherein when executed by one or more processors of a computing device, the at least one set of instructions causes the computing device to perform a method, the method comprising:
obtaining first information relating to an examination room where a movable medical device is located;
determining, based on the first information, at least one of environment information of the examination room or subject information relating to one or more subjects in the examination room, the one or more subjects including a target subject to be scanned; and
determining, based on the at least one of the environment information or the subject information, a scan protocol to be used in a medical scan of the target subject.
